(19) United States
(12) Patent Application Publication
(10) Pub. No.: US 2007/0135879 A1
(43) Pub. Date: Jun. 14, 2007

(54) Cylindrical device for delivering energy to tissue

(76) Inventors: Jon T. McIntyre, Newton, MA (US); Isaac Ostrovsky, Wellesley, MA (US); Ty Fairneny, Marlborough, MA (US)

Correspondence Address: FAY KAPLUN & MARCIN, LLP, 15O BROADWAY, SUITE 702, NEW YORK, NY, 10038, US

(21) Appl. No.: 11/297,785

(22) Filed: Dec. 8, 2005

(51) Int. Cl. A61F 7/12

(52) U.S. Cl. 607/113

(57) ABSTRACT

An energy delivery device comprises an elongated body sized and shaped for insertion into a body lumen via a naturally occurring body orifice and an acoustic energy element emitting energy around a circumference of a portion of the elongated body in combination with an electrical connection between the acoustic energy element and a power source to drive the acoustic energy element.

### BACKGROUND

Stress urinary incontinence (SUI) afflicts large numbers of women. One cause of SUI is believed to be the weakening of pelvic support structures, such as the fascia of the urethral wall, due to changes resulting from childbirth, pregnancy, menopause, etc.

A wide range of treatments of SUI are available depending on the severity of the condition and on the likely causes. However each of these approaches has limitations. Behavioral approaches (e.g., controlling diet and weight, muscular exercises, etc.) are often recommended only for mild cases and medication alone is generally ineffective while invasive surgical procedures entail significant patient discomfort and risk of complications.

### SUMMARY OF THE INVENTION

The present invention is directed to an energy delivery device comprising an elongated body sized and shaped for insertion into a body lumen via a naturally occurring body orifice and an acoustic energy element emitting energy around a circumference of a portion of the elongated body in combination with an electrical connection between the acoustic energy element and a power source to drive the acoustic energy element.

The present invention is further directed to a device for treating urinary incontinence, comprising an elongated body including a core portion having a diameter adapted to fit in the urethra and a hollow, substantially cylindrical crystal disposed on the core portion for generating acoustic energy in combination with a power source coupled to the crystal for activating the crystal.

### BRIEF DESCRIPTION OF DRAWINGS

FIG. 1 shows a cutaway view of an embodiment of an energy delivery device according to an embodiment of the present invention;

FIG. 2 shows a cross sectional side elevation view of a second embodiment of the energy delivery device according to the invention, comprising a segmented crystal;

FIG. 3 shows a cross sectional side elevation view of a third embodiment of the energy delivery device according to the invention, comprising a convex crystal; and

FIG. 4 shows a cross sectional side elevation view of a fourth embodiment of the energy delivery device according to the invention, comprising a concave crystal.

### DETAILED DESCRIPTION

The present invention may be further understood with reference to the following description and the appended drawings, wherein like elements are referred to with the same reference numerals. The present invention is related to medical devices used to access and treat tissue within a body lumen or cavity. In particular, the present invention relates to a system and method of treating tissue with acoustic energy to heat and shrink target tissue. Although the present invention is described in detail in regard to the treatment of Stress Urinary Incontinence, those skilled in the art will recognize that the device and method may be used in any number of procedures for heating and shrinking tissue and that the device and method are especially suited for use in endolumenal applications.

Less invasive endoscopic procedures have proven more effective in many cases than behavioral and medication based treatments. During these procedures, an endoscopic device is advanced to the tissues to be treated (e.g., muscles and ligaments supporting the bladder) through, for example, a body lumen accessed via a naturally occurring body orifice. The treatments are selected to enhance the ability of the target tissues to maintain the bladder in a desired position. For example, an endoscopic device may be inserted into the urethra to a desired position to treat a target portion of the urethral wall fascia.

In an exemplary embodiment of the invention, the target tissue is heated to denature collagen and shrink the tissue so that it draws the bladder to a desired position within the pelvis. Another possible response to this therapeutic heating is initiation of a fibroplastic response to stiffen the tissue structures, which may stabilize the area around the urethra. By properly selecting the location and amount of shrinking, it is possible to minimally invasively relieve the symptoms of even more advanced SUI.

According to exemplary embodiments of the present invention, a therapeutic device delivers acoustic energy inside a body cavity or lumen to heat target tissue below the surface tissues to a desired temperature. In one embodiment of the invention, a crystal generates acoustic energy and directs it radially outward toward the target tissue. The size and location of the region of tissue to be treated may be defined by selecting a length of the crystal, as well as by appropriately designing the crystal, as will be described in more detail below.

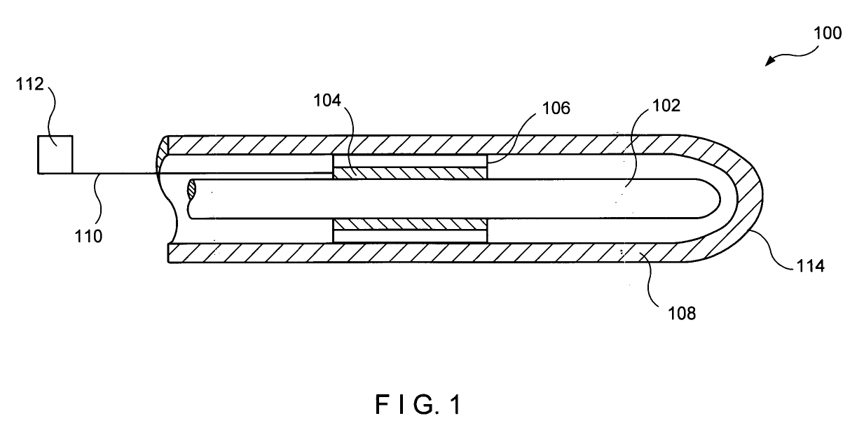

FIG. 1 shows a distal end of a device 100 includes a core 102 sized and shaped for insertion to a desired location in the urethra adjacent to a target portion of the fascia beneath the urethral wall. When the device 100 is in the target location, the user activates the device 100 to heat the target portion of the fascia, for example, up to about 70° C., to therapeutically heat the tissue. This reduces the compliance of the bladder neck and/or of the proximal urethra, so that the bladder is returned to a more correct position within the pelvis. Those skilled in the art will understand that the amount of shrinkage will depend on a variety of factors including the duration of the application of energy, the temperature to which the tissue is raised and the time that the tissue is held at the temperature. The core 102 according to this embodiment is formed as an elongated cylinder with an outer diameter selected to fit within a target body lumen (e.g., the urethra) and having stiffness sufficient to enable the device 100 to be pushed into the lumen without buckling. The core 102 is preferably formed of a biocompatible metal or plastic, as necessary to provide the desired mechanical properties of the energy delivery device 100.

An acoustic energy or piezo-electric element 104 receives an electrical energy and generates the acoustic energy used by the device 100 to provide thermal therapy to the target tissue. The acoustic energy element, also often called crystal, 104 is preferably a ceramic, which is electro statically poled to acquire piezo-electric properties. The common ceramic composition, which is used for the high intensity ultrasound applications, is Lead Zirconate Titanate, PZT. Other, not as common ceramics are Lithium Niobate, Potassium Niobate and Lead Nickel Niobate. The element, or crystal, is shaped to direct energy substantially symmetrically radially outward from the surface of the device 100 so that tissue surrounding the acoustic energy element 104 in all directions receives a substantially equal amount of energy. In the embodiment shown in FIG. 1, for example, the acoustic energy element 104 is substantially cylindrical to fit coaxially around the core 102. In addition, a length of the acoustic energy element 104 is preferably selected to optimize the delivery of acoustic energy to a target portion of tissue surrounding the body lumen in which the device 100 is inserted. For example, the length of the acoustic energy element 104 may be selected to be substantially equal to a length of the portion of tissue to be heated. In the example of FIG. 1, the length of the acoustic energy element 104 is selected to be substantially equal to a length of the portion of the fascia beneath the urethral wall to be treated.

According to the exemplary embodiment of the invention, the acoustic energy element 104 is connected to a power source 112 via, for example, an electrical connection 110. The power source 112 provides electric energy to the acoustic energy element 104, to generate acoustic energy to heat the target tissue to the target temperature to denature the collagen of this tissue in order to shrink and/or stiffen the target tissue to the desired level.

In a different embodiment according to the invention, the transfer of acoustic energy from the acoustic energy element 104 to the surrounding tissue is enhanced via an acoustic coupler 106 surrounding the acoustic energy element 104. The acoustic coupler 106 preferably includes a reservoir of fluid having properties enabling it to carry acoustic energy more efficiently than the air which would otherwise surround the acoustic energy element 104 within the core 102. In one embodiment, water is the fluid used as it carries sound energy more efficiently than air. In this embodiment, a substantially rigid outer shell 108 is disposed around the acoustic energy element 104, forming an annular gap between the core 102 and the outer shell 108. The gap may be filled with the acoustic coupler 106, as shown in FIG. 1. In another exemplary embodiment, the fluid of the acoustic coupler 106 flows along the gap 108, to set up a circulation to and from the distal tip 114 of the energy delivery device 100. Those skilled in the art will understand that any conventional method may be used to cause the fluid to flow around the acoustic energy element 104. This acoustic coupler 106 can also keep the surface of the device cool where it is adjacent to the tissue. This protects the tissue surface from thermal effects while the therapeutic ultrasound is penetrating deeper to the subsurface tissues.

In a different embodiment according to the invention, the outer shell 108 is formed of a compliant material which conforms to a shape of an inner surface of the lumen within which it is inserted. For example, an inflatable element such as a balloon may be used to form the outer shell 108 to accommodate variations in shape and size of the lumens from patient to patient. When this inflatable element is deflated it results in a overall lower cross-sectional area of the tip to facilitate insertion into, and passage through a lumen. Those skilled in the art will understand that the more completely a space between the acoustic energy element 104 and the target tissue is filled with the acoustic coupler 106, the more efficient the acoustic coupling between the device 100 and the target tissue will be.

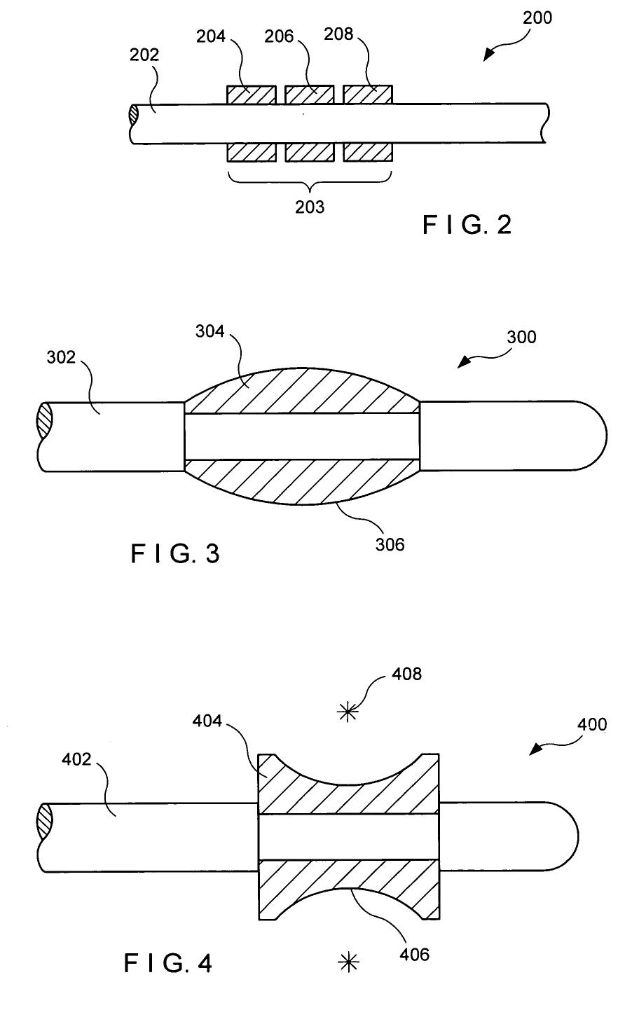

In some cases, an area of tissue that is not continuous is to be treated with the acoustic energy. It is preferred to avoid exposing non-targeted tissue to energy as energy applied to this non-targeted tissue is damaging to tissue while producing no therapeutic benefit and as this diverts energy from the targeted tissue. Accordingly, a device 200 according to a further embodiment of the invention employs a segmented acoustic energy element 203 with the segment s thereof being arranged with gaps therebetween. The gaps are located so that, when the device 200 is in a desired position adjacent to the target tissue, the gaps are adjacent to corresponding non-targeted portions of tissue while the segments of the acoustic energy element are located adjacent to targeted portions of tissue. As shown in FIG. 2, the device 200 includes an acoustic energy element 203 having segments 204, 206, 208 disposed on a core 202. As with the acoustic energy element 104, the segments 204-208 are powered from a power supply connected thereto in parallel or in series, depending on the requirements of the procedure. For example, those skilled in the art will understand that the segments 204, 206 and 208 are preferably connected so that they may be activated simultaneously or individually as the circumstances dictate. This segmented embodiment may also be used for contiguous tissue treatment and be more economical to manufacture as its uses many smaller crystals as opposed to one large crystal. One large crystal may be more fragile to handle and/or expensive to produce.

To carry out certain procedures, it may be beneficial to direct the energy of the acoustic energy element in a more controlled manner. For example, the acoustic energy element may be shaped to focus or to diffuse the acoustic energy generated therein, to achieve a particular therapeutic goal. FIG. 3 shows an exemplary embodiment of an energy delivery device 300, having a shaped acoustic energy element 304 adapted to target the delivery of acoustic energy according to a predetermined pattern. The exemplary device 300 is a direct contact probe, meaning that the acoustic energy element 304 fits snugly within the patient's body lumen, and is in direct contact with the wall of the lumen. The acoustic energy element 304 is formed to fit on a core portion 302, which provides a structural base for the acoustic energy element 304.

An outer surface 306 of the acoustic energy element 304 which according to this embodiment is a piezo-electric element is shaped to aim acoustic energy generated thereby. For example, the outer surface 306 may be convex, so that the sound energy from the acoustic energy element 304 is diffused outward from the energy delivery device 300. This design may be useful, for example, in applications where direct contact with the tissue to be treated is desired (possibly for treatment of BPH) and/or situations where a large portion of target tissue is to be heated, without accentuating the energy delivery to any one particular point. This allows a shorter length crystal to treat a larger area of tissue below the surface.

FIG. 4 shows another exemplary embodiment of an heating device 400, having a different shaped acoustic energy element 404 mounted on a core portion 402. In this example, the acoustic energy element 404 comprises a crystal with a concave outer surface 406 shaped to focus the generated acoustic energy at a desired distance away from the surface 406 of the acoustic energy element 404. Thus, energy from the acoustic energy element 404 is concentrated at a focal point 408, selected to coincide with a distance of the target tissue from the acoustic energy element 404 when the device 400 is in a desired position within a body lumen. In this manner, the acoustic energy of the acoustic energy element 404 is more efficiently delivered to the target tissue while reducing the intensity of energy delivered to intervening non-targeted tissues. In some cases it may be beneficial to combine different shapes of the outer surface of the crystal, thus including both flat, concave and convex portions or different degrees of concavity at different positions on the acoustic energy element 404. For example, if the depth of the target tissue varies along the length of the body lumen, the concavity of the acoustic energy element 404 may be varied along a length thereof while, if the depth of target tissue varies around a circumference of the body lumen, the concavity of the acoustic energy element 404 may be varied accordingly around a circumference of the device 400.

The exemplary embodiments of the present invention described above are directed primarily to an energy delivery device insertable in a patient's urethra to treat stress urinary incontinence. However, the same device may be used for other purposes as well, to carry out procedures that require the heating of selected regions of tissue near a body lumen or cavity. For example, the invention may be used to treat medical conditions such as fecal incontinence, prostate cancer, BPH and gastro-esophageal reflux disease (GERD). The ability of the devices according to the invention to heat targeted tissue may be advantageously used to shrink and otherwise cause a therapeutic effect to the targeted tissue.

The present invention was described with reference to specific exemplary embodiments. Those skilled in the art will understand that changes may be made in details, particularly in matters of shape, size, material and arrangement of parts. Accordingly, various modifications and changes may be made to the embodiments described herein. The specifications and drawings are, therefore, to be regarded in an illustrative rather than a restrictive sense.

What is claimed is:

1. An energy delivery device comprising:
an elongated body sized and shaped for insertion into a body lumen via a naturally occurring body orifice;
an acoustic energy element emitting energy around a circumference of a portion of the elongated body; and
an electrical connection between the acoustic energy element and a power source to drive the acoustic energy element.

2. The device according to claim 1, wherein the acoustic energy element includes a crystal vibrating in response to the power supplied thereto.

3. The device according to claim 2, wherein the crystal is a hollow substantially cylindrical crystal extending around a portion of the elongated body which, when the device is in a desired position within a body lumen, is adjacent to a target portion of tissue.

4. The device according to claim 1, wherein the acoustic energy element generates energy sufficient to denature collagen of in a target portion of tissue.

5. The device according to claim 1, further comprising an outer shell of the elongated body enclosing the acoustic energy element therein.

6. The device according to claim 5, wherein the outer shell is formed of a compliant material with an interior of the outer shell coupled to a source of an inflation fluid so that, when inflated, the outer shell conforms to a shape of an interior surface of a body lumen within which the device is received.

7. The device according to claim 5, wherein the outer shell comprises a cylindrical tube forming an annular space around the acoustic energy element.

8. The device according to claim 1, further comprising an acoustic coupler disposed around the acoustic energy element so that, when the device is received within a body lumen, the acoustic coupler is situated between the acoustic energy element and an inner wall of a body lumen.

9. The device according to claim 8, wherein the acoustic coupler comprises a liquid.

10. The device according to claim 9, wherein the liquid circulates within a space surrounding the acoustic energy element.

11. The device according to claim 1, wherein the acoustic energy element includes a plurality of crystal segments arranged so that, when the device is in a desired position within a body lumen, each segment is adjacent a corresponding target portion of tissue with gaps between the segments corresponding to locations not adjacent to target portions of tissue.

12. The device according to claim 11, wherein the segments are coupled to the power supply so that they may be activated individually of one another.

13. The device according to claim 11, wherein the segments are coupled to the power supply so that they may be activated simultaneously.

14. The device according to claim 11, wherein the segments are coupled to the power supply so that a first selected group of the segments may be activated independently of a second group of the segments.

15. The device according to claim 1, wherein the acoustic energy element comprises a crystal with a convex outer surface de-focusing acoustic energy generated thereby.

16. The device according to claim 1, wherein the acoustic energy element comprises a crystal with a concave outer surface focusing acoustic energy generated thereby at a predetermined distance from the device.

17. A device for treating urinary incontinence, comprising:
an elongated body including a core portion having a diameter adapted to fit in the urethra;
a hollow, substantially cylindrical crystal disposed on the core portion for generating acoustic energy; and
a power source coupled to the crystal for activating the crystal.

18. The device according to claim 17, further comprising an outer shell of the elongated body adapted to fit within the urethra, the outer shell enclosing the crystal.

19. The device according to claim 18, further comprising an acoustic coupler disposed between the cylindrical crystal and the outer shell.

20. The device according to claim 19, wherein the acoustic coupler is a fluid adapted to transmit acoustic energy to the outer shell.

21. The device according to claim 17, further comprising a fluid motion element circulating fluid around the crystal.

22. The device according to claim 17, wherein the crystal focuses the acoustic energy generated thereby to heat target tissue up to about 70° C.

23. The device according to claim 17, wherein an outer surface of the crystal follows a concavity selected to focus acoustic energy generated thereby on a selected portion of the urethral wall fascia.

24. The device according to claim 17, wherein the crystal comprises a convex outer surface diffusing acoustic energy generated thereby.

25. The device according to claim 17, wherein the crystal comprises a plurality of crystal segments, first and second ones of the segments being separated from one another by a gap.

26. The device according to claim 25, wherein the gaps are positioned so that, when the device is in a desired position relative to a target portion of tissue the gap is positioned adjacent to a non-targeted portion of tissue.

27. The device according to claim 26, wherein the crystal segments are coupled to the power source so that the segments can be individually powered.
